(19) United States
(12) Patent Application Publication
(10) Pub. No.: US 2007/0043127 A1
(43) Pub. Date: Feb. 22, 2007

(54) SYNTHESIS GAS CONVERSION AND NOVEL CATALYSTS FOR SAME

(75) Inventors: Jianhua Yao, Bartlesville, OK (US); James B. Kimble, Bartlesville, OK (US)

Correspondence Address: ConocoPhillips Company - I.P. Legal, P.O. BOX 2443, BARTLESVILLE, OK, 74005, US

(73) Assignee: Phillips Petroleum Company, Bartlesville, OK (US)

(21) Appl. No.: 11/552,011

(22) Filed: Oct. 23, 2006

Related U.S. Application Data
(62) Division of application No. 10/118,622, filed on Apr. 8, 2002.

(51) Int. Cl. B01J 23/72; C07C 27/26

(52) U.S. Cl. 518/726; 502/342; 502/346; 502/355

(57) ABSTRACT

A catalyst including a metal material and alumina, and a method of preparing such catalyst composition, are disclosed. A catalyst including a metal material, alumina, and thallium, and a method of preparing such catalyst composition are also disclosed. Each of these thus-obtained catalysts can be used for the conversion of synthesis gas into olefins.

### BACKGROUND OF THE INVENTION

The invention relates to catalyst compositions useful in synthesis gas (i.e., carbon monoxide and hydrogen) upgrading processes and methods for their production and use. In another aspect, this invention relates to processes for converting synthesis gas into olefins, with an increase in olefin selectivity and carbon monoxide conversion resulting from the conversion of such synthesis gas in the presence of such catalyst compositions.

It is known in the art to convert synthesis gas into olefins in the presence of catalysts. One concern with the use of catalysts in the conversion of synthesis gas to olefins is that not enough of the synthesis gas is converted. It is desirable to improve processes for the conversion of synthesis gas to olefms by increasing the percentage of carbon monoxide that is converted to olefins. It is also desirable to have a catalyst that is useful in producing significant quantities of butene conversion products.

### SUMMARY OF THE INVENTION

It is an object of this invention to provide improved catalyst compositions which when used in the conversion of synthesis gas result in increased carbon monoxide conversion and increased butene selectivity.

A further object of this invention is to provide methods for making improved catalyst compositions having such desirable properties to provide increased carbon monoxide conversion and increased butene selectivity.

Another object of this invention is to provide improved processes for the conversion of synthesis gas in which both the conversion of carbon monoxide and butene selectivity are increased.

In accordance with a first embodiment of this invention, the inventive catalyst composition consists of or consists essentially of (a) a metal material comprising a metal selected from the group consisting of zirconium, copper, zinc and combinations thereof; and (b) alumina.

In accordance with a second embodiment of this invention, the inventive catalyst composition can consist of or consist essentially of (a) a metal material comprising a metal selected from the group consisting of zirconium, copper, zinc and combinations thereof; (b) alumina; and (c) thallium.

A third embodiment of this invention includes a method comprising the steps of: (a) admixing a metal substance and an aluminum material to form a mixture thereof; (b) drying the mixture to form a dried mixture; and (c) calcining the dried mixture to form a calcined mixture consisting of or consisting essentially of alumina and a metal material comprising a metal selected from the group consisting of zirconium, copper, zinc, and combinations thereof.

A fourth embodiment of this invention includes the method of the third embodiment further comprising the steps of: (d) incorporating thallium into or onto the calcined mixture to form an incorporated mixture; and (e) drying the incorporated mixture to form a dried incorporated mixture consisting of or consisting essentially of alumina, thallium, and a metal material comprising a metal selected from the group consisting of zirconium, copper, zinc, and combinations thereof.

A fifth embodiment of this invention is a process comprising contacting synthesis gas with an inventive composition in a reaction zone under reaction conditions to thereby form a reaction product.

Other objects and advantages of the invention will become apparent from the detailed description and the appended claims.

### DETAILED DESCRIPTION OF THE INVENTION

In accordance with the first embodiment of the present invention, the inventive catalyst composition consists of or consists essentially of (a) a metal material comprising a metal selected from the group consisting of zirconium, copper, zinc and combinations thereof, and (b) alumina.

The metal material used in the inventive catalyst composition can be any metal material that is effective in the conversion of synthesis gas to olefms when contacted under reaction conditions with synthesis gas. Preferably, the metal of the metal material is zirconium. “Synthesis gas,” as employed herein, comprises, consists of, or consists essentially of a mixture of carbon monoxide and hydrogen.

Also preferably, the metal material is a metal oxide. Any metal oxide which can convert synthesis gas into olefins under reaction conditions can be used. More preferably, the metal oxide can be selected from the group consisting of zirconium oxide, copper oxide, and zinc oxide. Most preferably, the metal oxide is zirconium oxide.

Preferably, the metal material is present in an amount in the range of from about 10 to about 90 weight percent based on the total weight of the composition. More preferably, the metal material is present in an amount in the range of from about 50 to about 85 weight percent. Most preferably, the metal material is present in an amount in the range of from 65 to 85 weight percent.

Any suitable source of alumina can be employed in the composition, such as, for example, colloidal alumina solutions, hydrated aluminas, and peptized aluminas. Preferably, the alumina is present in the inventive composition in an amount in the range of from about 5 to about 90 weight percent based on the total weight of the composition. More preferably, the alumina is present in an amount in the range of from about 5 to about 50 weight percent. Most preferably, the alumina is present in an amount in the range of from 10 to 35 weight percent.

In accordance with the second embodiment of the present invention, the inventive catalyst composition can consist of or consist essentially of (a) a metal material comprising a metal selected from the group consisting of zirconium, copper, zinc and combinations thereof; (b) alumina; and (c) thallium. The metal material and alumina can be present in the same forms and weight percents as described above in the first embodiment. The thallium is preferably present in the composition in an amount in the range of from about 0.1 to about 15 weight percent, on an elemental thallium basis, based on the total weight of the composition. More preferably, the thallium is present in an amount in the range of from about 0.5 to about 10 weight percent, on an elemental thallium basis. Most preferably, the thallium is present in the composition in an amount in the range of from 1 to 7 weight percent, on an elemental thallium basis.

In accordance with the third embodiment of the present invention, the inventive catalyst composition can be produced by the following method comprising: (a) admixing a metal substance and an aluminum material to form a mixture thereof; (b) drying the mixture to form a dried mixture; and (c) calcining the dried mixture to form a calcined mixture consisting of or consisting essentially of alumina and a metal material comprising a metal selected from the group consisting of zirconium, copper, zinc, and combinations thereof.

The admixing of step (a) can include combining a metal substance and an aluminum material in appropriate proportions by any suitable method or manner which provides for the intimate mixing of such components to thereby provide a substantially homogeneous mixture thereof comprising a metal substance and an aluminum material. Any suitable means for admixing the components of the inventive catalyst composition can be used to achieve the desired dispersion of such components. Examples of suitable admixing means include, but are not limited to, mixing tumblers, stationary shelves or troughs, Eurostar mixers, which are of the batch or continuous type, impact mixers, and the like.

Any suitable metal substance can be used to produce the inventive composition. Preferably, the metal substance comprises a metal selected from the group consisting of zirconium, copper, and zinc. Preferably, the metal is zirconium.

The metal substance can be formed by any suitable manner known in the art. Preferably, the metal substance is formed by admixing ammonia and a metal-containing compound. Suitable metal-containing compounds include, but are not limited to metal nitrates, metal carbonates, metal nitrites, metal fluorides, metal chlorides, metal bromides, metal acetates, metal isopropoxides, metal butoxides, and combinations of two or more thereof. Most preferably, the metal substance is formed by admixing ammonia and a metal nitrate.

Also preferably, the metal substance used for the production of the catalyst composition is a metal hydroxide. More preferably the metal hydroxide is selected from the group consisting of zirconium hydroxide, copper hydroxide, zinc hydroxide and combinations thereof. Most preferably the metal hydroxide is zirconium hydroxide.

The weight percent of the metal material present in the calcined mixture is generally in the range of from about 10 to about 90 weight percent, preferably from about 50 to about 85 weight percent, and most preferably from 65 to 85 weight percent based on the total weight of the calcined mixture.

Any suitable aluminum material can be used in the method to produce the inventive catalyst composition. The aluminum material can be formed by any suitable manner known in the art. Preferably, the aluminum material is formed by admixing ammonia and an aluminum-containing compound. Suitable aluminum-containing compounds can include, but are not limited to aluminum nitrates, aluminum carbonates, aluminum nitrites, aluminum fluorides, aluminum chlorides, aluminum bromides, aluminum acetates, aluminum isopropoxides, aluminum butoxides, and combinations of two or more thereof. Most preferably, the aluminum material is formed by admixing ammonia and an aluminum nitrate.

Preferably, the aluminum material is aluminum hydroxide.

The weight percent of the alumina present in the calcined mixture is generally in the range of from about 5 to about 90 weight percent, preferably in the range of from about 5 to about 50 weight percent, and most preferably in the range of from 10 to 35 weight percent based on the total weight of the calcined mixture.

Drying conditions for step (b) include a drying temperature in the range of from about 25° C. to about 200° C. More preferably, the drying temperature is in the range of from about 50° C. to about 150° C., and most preferably the drying temperature is in the range of from 100° C. to 150° C. Any drying method known to one skilled in the art such as, for example, air drying, heat drying, and the like and combinations thereof can be used. Preferably, heat drying is used.

Calcining conditions in step (c) include a calcining temperature in the range of from about 250° C. to about 800° C. More preferably the calcining temperature is in the range of from about 300° C. to about 700° C., and most preferably the calcining temperature is in the range of from 400° C. to 600° C. The calcination occurs in the presence of air.

In accordance with the fourth embodiment of the present invention, the calcined mixture of the third embodiment can be modified by: (d) incorporating thallium into or onto the calcined mixture to form an incorporated mixture; and (e) drying the incorporated mixture to form a dried incorporated mixture consisting of or consisting essentially of alumina, thallium, and a metal material comprising a metal selected from the group consisting of zirconium, copper, zinc, and combinations thereof.

The thallium can be incorporated into or onto the calcined mixture in step (d) by any suitable manner known in the art. Preferably, the thallium, being in the form of thallous nitrate, is impregnated into or onto the calcined mixture. The thallous nitrate can be impregnated into or onto the calcined mixture by any suitable means or method known in the art. Preferably, the thallous nitrate can be impregnated into or onto the calcined mixture by using any standard incipient wetness technique (i.e. essentially completely or partially filling the pores of a substrate material with a solution of the incorporating elements) for impregnating a substrate.

Any suitable form of thallium can be used in the method to produce the inventive catalyst composition. Preferably, the thallium is in the form of thallous nitrate. The weight percent of the thallium present in the dried incorporated mixture is generally in the range of from about 0.1 to about 15 weight percent, preferably from about 0.5 to about 10 weight percent, and most preferably from 1 to 7 weight percent, on an elemental thallium basis, based on the total weight of the dried incorporated mixture.

The weight percent of the metal material present in the dried incorporated mixture is generally in the range of from about 10 to about 90 weight percent, preferably from about 50 to about 85 weight percent, and most preferably from 65 to 85 weight percent based on the total weight of the dried incorporated mixture.

The weight percent of the alumina present in the dried incorporated mixture is generally in the range of from about 5 to about 90 weight percent, preferably in the range of from about 5 to about 50 weight percent, and most preferably in the range of from 10 to 35 weight percent based on the total weight of the dried incorporated mixture.

The drying conditions for step (e) in the fourth embodiment are the same as those for step (b) of the third embodiment, as described above.

In accordance with the fifth embodiment, this invention further provides a process comprising, consisting of, or consisting essentially of contacting synthesis gas with a composition comprising alumina and a metal material wherein the metal is selected from the group consisting of zirconium, copper, zinc, and combinations of two or more thereof in a reaction zone and under reaction conditions to thereby form a reaction product. The composition can further comprise thallium. The composition can be selected from those described above in embodiments one, two, three, and four.

This process produces a product comprising at least one olefin containing in the range of from 2 to 6 carbon atoms per molecule. Preferably the reaction product comprises at least one olefin containing in the range of from 2 to 4 carbon atoms per molecule.

The contacting step can be operated as a batch process step or, preferably, as a continuous process step. In the latter operation, a solid catalyst bed, or a moving catalyst bed, or a fluidized catalyst bed can be employed. Any of these operational modes have advantages and disadvantages and those skilled in the art can select the one most suitable for a particular catalyst.

The contacting step is preferably carried out within a reaction zone, which contains the inventive catalyst composition, and under reaction conditions that suitably promote the formation of olefins from at least a portion of the synthesis gas. The reaction temperature of the contacting step is generally in the range of from about 200° C. to about 600° C., preferably from about 300° C. to about 400° C., and most preferably from 350° C. to 400° C. The contacting pressure can generally range from about 100 psig to about 800 psig, preferably, from about 150 psig to about 400 psig, and most preferably, 200 psig to 300 psig.

The process can be carried out in the presence of one or more inert diluents. If a diluent is used, preferably the diluent is nitrogen.

### EXAMPLES

The following examples are intended to be illustrative of the present invention and to teach one of ordinary skill in the art to make and use the invention. These examples are not intended to limit the invention in any way.

Examples I through VI compare zirconia/alumina catalysts. In Example I, the catalyst is formed by co-precipitating zirconia and alumina salts. In Examples III and V, the catalyst is formed by precipitating zirconia and alumina salts separately and then mixing the two precipitates together, as disclosed in this invention. The synthesis gas conversion in Examples II, IV, and VI use the catalysts of Examples I, III, and V, respectively.

### Example I

### Control

A 30 gram quantity of zirconyl nitrate hydrate was added to 33 grams of aluminum nitrate nonahydrate. This mixture was then dissolved in about 600 mL of water. A 15% ammonia solution was prepared by mixing 170 mL of concentrated ammonia solution and 170 mL water. The zirconyl/aluminum solution and the ammonia solution were then added to a beaker simultaneously while stirring at room temperature. The solution was continuously stirred for about 2 hours and was then left to sit overnight. The precipitate that had formed was then filtered, washed with water, and extruded. The composition was then dried overnight at 120° C. and then calcined for two hours at a temperature of 500° C.

### Example II

### Control

A 7 gram quantity of the composition prepared in Example I was placed into a stainless steel tube reactor (inner diameter: about 0.5 inch). The steel reactor tube was heated to about 400° C. The reactor pressure was 250 psig. A carbon monoxide/hydrogen feed was introduced to the reactor tube at a flow rate of about 33.2 mL/minute. This feed was allowed to flow through the reactor tube for four hours before a product was sampled and tested. The carbon monoxide conversion and olefin selectivity are shown in Table I.

### Example III

### Inventive

A 30 gram quantity of zirconyl nitrate hydrate was dissolved in 350 mL of water. A 170 mL quantity of a 15% ammonia aqueous solution (prepared as described in Example I) was quickly added to the zirconyl solution while stirring at room temperature. The solution was stirred for 1 hour, and then left to sit out overnight. The precipitate that had formed was then filtered and washed with water. Meanwhile, 33 grams of aluminum nitrate nonahydrate were dissolved in 250 mL of water. A 170 mL quantity of a 15% ammonia aqueous solution was quickly added to the aluminum solution while stirring at room temperature. The solution was stirred for 1 hour, and then left to sit out overnight. The precipitate that had formed was then filtered and washed with water.

The zirconyl precipitate and the aluminum precipitate were then mixed together in a beaker with about 100 mL of water. This solution was then stirred for 1 hour. The solution was then filtered, washed with water, and extruded. The solution was then dried overnight at a temperature of 120° C. and calcined for two hours at a temperature of 500° C.

### Example IV

### Inventive

About 5.6 grams of the composition prepared in Example III were placed into a stainless steel tube reactor (inner diameter: about 0.5 inch). The steel reactor tube was heated to about 400° C. The reactor pressure was 250 psig. A carbon monoxide/hydrogen feed was introduced to the reactor tube at a flow rate of about 33.2 mL/minute. This feed was allowed to flow through the reactor tube for three hours before a product was sampled and tested. The carbon monoxide conversion and olefin selectivity are shown in Table I.

### Example V

### Inventive

A 30 gram quantity of zirconyl nitrate hydrate was dissolved in 300 mL of water. A 170 mL quantity of a 15% ammonia aqueous solution (prepared as described in Example I) was added dropwise to the zirconyl solution while stirring at room temperature. The solution was stirred for 1 hour, and then left to sit out overnight. The precipitate that had formed was then filtered and washed with water. Meanwhile, 33 grams of aluminum nitrate nonahydrate were dissolved in 250 mL of water. A 170 mL quantity of a 15% ammonia aqueous solution was quickly added to the aluminum solution while stirring at room temperature. The solution was stirred for about 2 hours, and then left to sit out overnight. The precipitate that had formed was then filtered and washed with water.

The zirconyl precipitate and the aluminum precipitate were then mixed together in a beaker with about 100 mL of water. This solution was then stirred for 1 hour. The solution was then filtered, washed with water, and extruded. The solution was then dried overnight at a temperature of 120° C. and calcined for two hours at a temperature of 500° C.

### Example VI

### Inventive

A 7 gram quantity of the composition prepared in Example V was placed into a stainless steel tube reactor (inner diameter: about 0.5 inch). The steel reactor tube was heated to about 400° C. The reactor pressure was 250 psig. A carbon monoxide/hydrogen feed was introduced to the reactor tube at a flow rate of about 33.2 mL/minute. This feed was allowed to flow through the reactor tube for three hours before a product was sampled and tested. The carbon monoxide conversion and olefin selectivity are shown in Table I.

Table I shows that catalysts formed by precipitating zirconia and alumina (Examples III and V) separately display better carbon monoxide conversion than the catalyst formed by co-precipitating zirconia and alumina (as in Example I).

TABLE I

| | CO | | | | |
|---|---|---|---|---|---|
| | Conversion | Olefin Selectivity (wt. %) | | | |
| Catalyst | (%) | C₂ | C₃ | C₄ | Total |
| Example I (Control) | 16.5 | 24.5 | 15.3 | 24.8 | 64.6 |
| Example III (Inventive) | 22.3 | 27.7 | 15.6 | 21.1 | 64.4 |
| Example V (Inventive) | 21.6 | 27.5 | 16.6 | 23.3 | 67.4 |

Examples VII through X compare a zirconia/alumina catalyst (prepared in Example VII) with a zirconia/alumina/thallium catalyst (prepared in Example IX).

### Example VII

### Inventive

A 50 gram quantity of zirconyl nitrate hydrate was dissolved in 500 mL of water. A 280 mL quantity of a 15% ammonia aqueous solution (prepared as described in Example I) was added dropwise to the zirconyl solution while stirring at room temperature. The solution was stirred for 1 hour, and then left to sit out overnight. The precipitate that had formed was then filtered and washed with water. Meanwhile, 33 grams of aluminum nitrate nonahydrate were dissolved in 250 mL of water. A 170 mL quantity of a 15% ammonia aqueous solution was quickly added to the aluminum solution while stirring at room temperature. The solution was stirred for 1 hour, and then left to sit out overnight. The precipitate that had formed was then filtered and washed with water.

The zirconyl precipitate and the aluminum precipitate were then mixed together in a beaker with about 100 mL of water. This solution was then stirred for about 2 hours. The solution was then filtered, washed with water, and extruded. The solution was then dried overnight at a temperature of 120° C. and calcined for two hours at a temperature of 500° C.

### Example VIII

### Inventive

A 7 gram quantity of the composition prepared in Example VII was placed into a stainless steel tube reactor (inner diameter: about 0.5 inch). The steel reactor tube was heated to about 400° C. The reactor pressure was 250 psig. A carbon monoxide/hydrogen feed was introduced to the reactor tube at a flow rate of about 33.2 mL/minute. This feed was allowed to flow through the reactor tube for 3 more hours before a product was measured. The carbon monoxide conversion and olefin selectivity are shown in Table II.

### Example IX

### Inventive

A thallium solution was prepared by dissolving 0.54 grams of thallous nitrate in 6.5 mL of water. An 8.3 gram quantity of the composition prepared in Example VII was then added to this solution. The mixture was then allowed to sit at room temperature for a half hour and was then dried overnight at a temperature of 120° C.

### Example X

### Inventive

A 7 gram quantity of the composition prepared in Example IX was placed into a stainless steel tube reactor (inner diameter: about 0.5 inch). The steel reactor tube was heated to about 400° C. The reactor pressure was 250 psig. A carbon monoxide/hydrogen feed was introduced to the reactor tube at a flow rate of about 33.2 mL/minute. This feed was allowed to flow through the reactor tube for three hours before a product was sampled and tested. The carbon monoxide conversion and olefin selectivity are shown in Table II.

As is evident from Table II, the catalyst containing thallium (as prepared in Example IX) demonstrates a much higher selectivity for butenes than a zirconia/alumina catalyst not containing thallium.

TABLE II

| | CO | | | |
|---|---|---|---|---|
| | Conversion | Olefin Selectivity (wt. %) | | |
| Catalyst | (%) | C₂ | C₃ | C₄ |
| Example VII (Inventive; | 21.9 | 24.9 | 15.1 | 24.3 |
| without thallium) | | | | |
| Example IX (Inventive; | 10.5 | 7.1 | 3.0 | 43.4 |
| with thallium) | | | | |

What is claimed is:

1-31. (canceled)

32. A method comprising the steps of:
(a) admixing a metal substance and an aluminum material to form a mixture thereof;
(b) drying said mixture to from a dried mixture; and
(c) calcining said dried mixture to form a calcined mixture consisting essentially of alumina and a metal material comprising a metal selected from the group consisting of zirconium, copper, zinc, and combinations thereof.

33. A method in accordance with claim 32 wherein said metal substance is formed by admixing ammonia and a metal-containing compound selected from the group consisting of metal nitrates, metal carbonates, metal nitrites, metal fluorides, metal chlorides, metal bromides, metal acetates, metal isopropoxides, metal butoxides, and combinations of two or more thereof.

34. A method in accordance with claim 33 wherein said metal-containing compound is a metal nitrate.

35. A method in accordance with claim 32 wherein said aluminum material is formed by admixing ammonia and an aluminum-containing compound selected from the group consisting of aluminum nitrates, aluminum carbonates, aluminum nitrites, aluminum fluorides, aluminum chlorides, aluminum bromides, aluminum acetates, aluminum isopropoxides, aluminum butoxides, and combinations of two or more thereof.

36. A method in accordance with claim 35 wherein said aluminum-containing compound is an aluminum nitrate.

37. A method in accordance with claim 32 wherein said metal substance comprises a metal selected from the group consisting of zirconium, copper, and zinc.

38. A method in accordance with claim 32 wherein said metal material is present in said calcined mixture in an amount in the range of from about 10 to about 90 weight percent based on the total weight of said calcined mixture.

39. A method in accordance with claim 32 wherein said metal material is present in said calcined mixture in an amount in the range of from about 50 to about 85 weight percent based on the total weight of said calcined mixture.

40. A method in accordance with claim 32 wherein said metal material is present in said calcined mixture in an amount in the range of from 65 to 85 weight percent based on the total weight of said calcined mixture.

41. A method in accordance with claim 32 wherein said alumina is present in said calcined mixture in an amount in the range of from about 5 to about 90 weight percent based on the total weight of said calcined mixture.

42. A method in accordance with claim 32 wherein said alumina is present in said calcined mixture in an amount in the range of from about 5 to about 50 weight percent based on the total weight of said calcined mixture.

43. A method in accordance with claim 32 wherein said alumina is present in said calcined mixture in an amount in the range of from 10 to 35 weight percent based on the total weight of said calcined mixture.

44. A method in accordance with claim 32 wherein said drying in step (b) includes a drying temperature in the range of from about 25° C. to about 200° C.

45. A method in accordance with claim 32 wherein said drying in step (b) includes a drying temperature in the range of from about 50° C. to about 150° C.

46. A method in accordance with claim 32 wherein said drying in step (b) includes a drying temperature in the range of from 100° C. to 150° C.

47. A method in accordance with claim 32 wherein said calcining in step (c) includes a calcining temperature in the range of from about 250° C to about 800° C.

48. A method in accordance with claim 32 wherein said calcining in step (c) includes a calcining temperature in the range of from about 300° C. to about 700° C.

49. A method in accordance with claim 32 wherein said calcining in step (c) includes a calcining temperature in the range of from 400° C. to 600° C.

50. A method in accordance with claim 32 wherein said aluminum material is aluminum hydroxide.

51. A method in accordance with claim 32 wherein said metal substance is a metal hydroxide.

52. A method in accordance with claim 51 wherein said metal hydroxide is zirconium hydroxide.

53. A method in accordance with claim 51 wherein said metal hydroxide is copper hydroxide.

54. A method in accordance with claim 51 wherein said metal hydroxide is zinc hydroxide.

55. A method in accordance with claim 32 further comprising the steps of:
(d) incorporating thallium into or onto said calcined mixture to form an incorporated mixture; and
(e) drying said incorporated mixture to form a dried incorporated mixture consisting essentially of alumina, thallium, and a metal material comprising a metal selected from the group consisting of zirconium, copper, zinc, and combinations thereof.

56. A method in accordance with claim 55 wherein said thallium in step (d) is in the form of thallous nitrate.

57. A method in accordance with claim 55 wherein said thallium in step (d) is incorporated by impregnation.

58. A method in accordance with claim 55 wherein said thallium is present in said dried incorporated mixture in an amount in the range of from about 0.1 to about 15 weight percent, on an elemental thallium basis, based on the total weight of said dried incorporated mixture.

59. A method in accordance with claim 55 wherein said thallium is present in said dried incorporated mixture in an amount in the range of from about 0.5 to about 10 weight percent, on an elemental thallium basis, based on the total weight of said dried incorporated mixture.

60. A method in accordance with claim 55 wherein said thallium is present in said dried incorporated mixture in an amount in the range of from 1 to 7 weight percent, on an elemental thallium basis, based on the total weight of said dried incorporated mixture.

61. A method in accordance with claim 55 wherein said drying in step (e) includes a drying temperature in the range of from about 25° C. to about 200° C.

62. A method in accordance with claim 55 wherein said drying in step (e) includes a drying temperature in the range of from about 50° C. to about 150° C.

63. A method in accordance with claim 55 wherein said drying in step (e) includes a drying temperature in the range of from 100° C. to 150° C.

64. A method in accordance with claim 55 wherein said aluminum material is aluminum hydroxide.

65. A method in accordance with claim 55 wherein said metal substance is a metal hydroxide.

66. A method in accordance with claim 55 wherein said metal hydroxide is zirconium hydroxide.

67. A method in accordance with claim 65 wherein said metal hydroxide is copper hydroxide.

68. A method in accordance with claim 65 wherein said metal hydroxide is zinc hydroxide.

69-72. (canceled)

73. A process comprising:
contacting synthesis gas with a composition comprising alumina and a metal material wherein said metal is selected from the group consisting of zirconium, copper, zinc and combinations thereof in a reaction zone and under reaction conditions to thereby form a reaction product.

74. A process comprising contacting synthesis gas with a composition in a reaction zone and under reaction conditions to thereby form a reaction product, wherein said composition is the composition formed by the process of claim 32.

75. A process comprising contacting synthesis gas with a composition in a reaction zone and under reaction conditions to thereby form a reaction product, wherein said composition is the composition formed by the process of claim 52.

76. A process in accordance with claim 73 wherein said reaction product comprises at least one olefin containing in the range of from 2 to 6 carbon atoms per molecule.

77. A process in accordance with claim 73 wherein said reaction product comprises at least one olefin containing in the range of from 2 to 4 carbon atoms per molecule.

78. A process in accordance with claim 73 wherein said reaction conditions include a reaction temperature in the range of from about 200° C. to about 600° C.

79. A process in accordance with claim 73 wherein said reaction conditions include a reaction temperature in the range of from about 300° C. to about 400° C.

80. A process in accordance with claim 73 wherein said reaction conditions include a reaction temperature in the range of from 350° C. to 400° C.

81. A process in accordance with claim 73 wherein said reaction conditions include a reaction pressure in the range of from about 100 psig to about 800 psig.

82. A process in accordance with claim 73 wherein said reaction conditions include a reaction pressure in the range of from about 150 psig to about 400 psig.

83. A process in accordance with claim 73 wherein said synthesis gas comprises carbon monoxide and hydrogen.

84. A process comprising:
contacting synthesis gas with a composition comprising alumina, thallium, and a metal material wherein said metal is selected from the group consisting of zirconium, copper zinc and combinations thereof in a reaction zone and under reaction conditions to thereby form a reaction product.

85. A process comprising contacting synthesis gas with a composition in a reaction zone and under reaction conditions to thereby form a reaction product, wherein said composition is the composition formed by the process of claim 55.

86. A process comprising contacting synthesis gas with a composition in a reaction zone and under reaction conditions to thereby form a reaction product, wherein said composition is the composition formed by the process of claim 66.

87. A process in accordance with claim 84 wherein said reaction product is in the form of at least one olefin containing in the range of from 2 to 6 carbon atoms per molecule.

88. A process in accordance with claim 84 wherein said reaction product is in the form of at least one olefin containing in the range of from 2 to 4 carbon atoms per molecule.

89. A process in accordance with claim 84 wherein said reaction conditions include a reaction temperature in the range of from about 200° C. to about 600° C.

90. A process in accordance with claim 84 wherein said reaction conditions include a reaction temperature in the range of from about 300° C. to about 400° C.

91. A process in accordance with claim 84 wherein said reaction conditions include a reaction temperature in the range of from 350° C. to 400° C.

92. A process in accordance with claim 84 wherein said reaction conditions include a reaction pressure in the range of from about 100 psig to about 800 psig.

93. A process in accordance with claim 84 wherein said reaction conditions include a reaction pressure in the range of from about 150 psig to about 400 psig.

94. A process in accordance with claim 84 wherein said synthesis gas comprises carbon monoxide and hydrogen.
